(19) United States
(12) Patent Application Publication
(10) Pub. No.: US 2010/0222312 A1
(43) Pub. Date: Sep. 2, 2010

(54) DELAYED-RELEASE GLUCOCORTICOID TREATMENT OF ASTHMA

(75) Inventors: Stephan WITTE, Schriesheim (DE); Achim Schäffler, Beerfelden (DE)

Correspondence Address: ROTHWELL, FIGG, ERNST & MANBECK, P.C., 1425 K STREET, N.W., SUITE 800, WASHINGTON, DC, 20005, US

(73) Assignee: NITEC PHARMA AG, Reinach (CH)

(21) Appl. No.: 12/693,565

(22) Filed: Jan. 26, 2010

Related U.S. Application Data
(60) Provisional application No. 61/147,165, filed on Jan. 26, 2009.

(51) Int. Cl. A61K 31/58; A61K 31/573; A61P 11/06

(52) U.S. Cl. 514/174; 514/176; 514/179; 514/180

(57) ABSTRACT

The present invention refers to the treatment of asthma by administering a delayed-release dosage form of a glucocorticoid to a subject in need thereof.

The present invention refers to the treatment of asthma, particularly to the treatment of severe uncontrolled asthma with nocturnal symptoms by administering a delayed-release dosage form of a glucocorticoid to a subject in need thereof.

### BACKGROUND OF THE INVENTION

Asthma is a chronic inflammatory disorder of the airways which is associated with airway hyperresponsiveness that leads to recurrent symptomatic episodes of wheezing, breathlessness, chest tightness, and coughing, particularly at night or in the early morning. These episodes are associated with widespread, but variable, airflow obstruction within the lung that is often reversible either spontaneously or with treatment (Global Initiative for Asthma, GINA 2008).

A typical feature of asthma is the circadian nature of asthma symptoms, with most of them occurring at night or in the early morning hours. Even in healthy individuals, lung function has been shown to fluctuate over a 24-hour period, with poorest lung function during the night around 4 am. However in patients with asthma these fluctuations are more pronounced.

More than 70% of 7729 studied patients with asthma have been shown to frequently experience nocturnal symptoms at least once week and more than 25% of patients, who rates their asthma as “mild” reported nocturnal awakenings due to asthma symptoms every night (Turner-Warwick 1988). Another study of 3129 patients showed that more than 90% of dyspnoeic episodes occurred between midnight and 7 am with 4 am being the time of peak symptom frequency (Dethefsen 1985). Nocturnal asthma symptoms are also related with asthma mortality, as the majority of asthma related deaths occur between midnight and 8 am (Sutherland 2005 and references therein).

Nocturnal symptoms and nocturnal awakenings because of asthma symptoms is such a typical characteristic of asthma that it has become a standard diagnostic criterion for the disease. The absence of nocturnal symptoms and nocturnal awakenings is required for the classification of asthma as “controlled”. (GINA 2008).

The mechanisms responsible for the circadian variation of asthma symptoms are complex and involve the HPA axis and it was suggested that adrenal responsiveness to corticotrophin is blunted by the underlying chronic inflammation (Sutherland 2005).

Most patients, with mild to moderate asthma are usually well controlled with inhaled glucocorticoids and β₂-adrenoreceptor agonists. However, approx. 10% of patients with asthma cannot achieve acceptable control of their asthma symptoms despite being treated with high doses of inhaled glucocorticoids and long acting β₂-adrenoreceptor agonists. This subset of patients has greatest impairment of their lifestyles and account for the majority of asthma related healthcare costs (Holgate 2006 and references therein)

For such patients with severe uncontrolled asthma, long term therapy with oral glucocorticoids in addition to their standard therapy may be required (GINA 2007). The glucocorticoid used is primarily prednisone, prednisolone or methylprednisolone. The accepted standard regimen for administration of oral glucocorticoids is administration as a single dose in the morning.

Beam et al. (Beam 1992) reported that an administration of oral prednisone at 3 pm could be favourable.

Niphadkar et al (2005) reported that the time-point of administration of inhaled ciclesonide is not of importance. AM vs PM inhalation did not reveal any difference in the effectiveness of glucocorticoids. Zetterstrom et al (2008) confirmed the findings from Niphadkar that the time-point of administration of the glucocorticoid does not influence the efficacy. He compared measured day and night time symptoms of asthma patients after the administration of 400 μg inhaled Mometasone furoate DPI.

### Delayed-Release Prednisone Tablets

U.S. Pat. No. 5,792,476 describes a pharmaceutical composition for peroral administration for rheumatoid arthritis, which comprises a glucocorticoid as active ingredient and which leads to release in the small intestine. The composition is a granulate which is laminated with an inner layer which is resistant to a pH of 6.8, and with an outer layer which is resistant to a pH of 1.0.

U.S. Pat. No. 6,488,960 describes a pharmaceutical dosage form for controlled release of corticoids, reference being made to the formulations described in U.S. Pat. No. 5,792,476.

WO 01/08421 describes a tablet having a core which is coated by at least two layers, one of which completely encloses the other. The coating layers can be produced by spray coating and/or pressing.

WO 01/68056 discloses a pharmaceutical preparation having a release profile with a time delay, comprising a core and at least one hydrophilic or lipophilic coating surrounding the core, where the coating is slowly swollen, dissolved, eroded or changed in its structure in another way through the water present in the release medium, so that the core or parts of the core become accessible to the release medium. The coating may be formed for example as pressed coating.

WO 02/072034 discloses a pharmaceutical dosage form for delayed release, having a core which comprises as active ingredient a glucocorticoid and a material which brings about delayed release and includes at least one natural or synthetic gum.

WO 2004/093843 discloses a tablet with a specific core geometry to release the active ingredient in a specific delayed release manner.

WO 2006/027266 discloses a pharmaceutical dosage form with site- and time controlled gastrointestinal release of an active agent, particularly a corticosteroid. The pharmaceutical dosage form is preferably a coated tablet having a core comprising the corticosteroid and a swellable/disintegration adjuvant, and an inert outer coating. The coating is compressed at a pressure chosen to result in the release of the corticosteroid at a predetermined position in the gastrointestinal tract.

WO 2008/015018 discloses the long-term use of delayed release dosage form of glucocorticoids for the treatment of rheumatic diseases.

### SUMMARY OF THE INVENTION

The present inventors have carried out a clinical pilot study in order to test the efficacy of therapy with delayed-release prednisone tablets compared to therapy with conventional standard immediate-release tablets. It was found that asthma therapy with the delayed-release prednisone tablets shows a surprisingly increased efficacy compared to the therapy with conventional standard immediate-release prednisone tablets.

This invention provides evidence that a release of the glucocorticoid at 2 am via a delayed release tablet which is administered at bed-time is superior to a standard tablet given in the morning. Superiority is defined by less symptoms, such as nocturnal awakenings, improved quality of life and less usage of rescue medication. This finding is surprising as the literature teaches that the timed-point of administration should be ideally 3 PM (Beam 1992) or seemed to be less important (Niphadkar 2005, Zetterström 2008).

Thus, a first aspect of the invention refers to the use of a delayed-release dosage form of a corticosteroid for the manufacture of a medicament for the treatment of asthma.

The invention further refers to a method for the treatment of a patient suffering from asthma, particularly from uncontrolled asthma, with nocturnal awakenings, which comprises administering to said patient an effective amount of a glucocorticoid contained in a delayed-release dosage form, wherein said treatment is administered once daily.

The invention further refers to a method for the treatment of an asthma patient having circadian fluctuations in Interleukin 6 levels due to underlying inflammation, which comprises administering to said patient an effective amount of a glucocorticoid contained in a delayed-release dosage form, wherein said treatment is administered once daily, and wherein said treatment is administered such that the glucocorticoid is released at or before the time when the patient's Interleukin 6 level is at a daily peak.

The invention further refers to a method for the treatment of a patient suffering from asthma, which comprises administering, to said patient an effective amount of a glucocorticoid contained in a delayed-release dosage form, wherein the pharmacokinetics after administering of said dosage form are equivalent to the pharmacokinetics after administering an immediate release dosage form, wherein the pharmacokinetics include an equivalent Cmax, an equivalent AUC and/or an equivalent tmax-tlag.

The invention encompasses the use of delayed-release glucocorticoids in different types of asthma, such as bronchial asthma, e.g. allergic asthma, infection-associated asthma, mixed-form asthma (e.g. allergic asthma exaggerated by an infection), drug, e.g. analgesic induced asthma or exercise induced asthma or cardial asthma. It includes the treatment of asthma that shows reversibility upon the administration of a bronchodilator as well as the treatment of asthma not showing reversibility after administration of a bronchodilator. It also includes the treatment of asthma which is uncontrolled, particularly the treatment of severe uncontrolled asthma with nocturnal symptoms.

Preferred is the use of delayed-release glucocorticoids for the treatment of severe asthma as defined by the requirement for continued treatment with oral glucocorticoids and/or for the treatment of nocturnal asthma as defined by frequent nocturnal awakenings due to asthma i.e. at least one, at least two or at least three nocturnal awakenings per week on average. Especially preferred is the use of delayed-release glucocorticoids in the treatment of severe nocturnal asthma as defined by frequent nocturnal awakenings and the requirement for continued treatment with oral glucocorticoids.

A further aspect of the invention refers to the use of a delayed-release dosage form of a glucocorticoid for the manufacture of a medicament for the treatment of asthma in

(i) patients with severe diseases,
(ii) patients with moderate diseases,
(iii) patients with uncontrolled disease duration,
(iv) patients with nocturnal symptoms of the disease,
(v) patients with short disease duration (<2 years),
(vi) patients with mid-term disease duration (2-5 years) or
(vii) patients with long-lasting disease duration (>5 years).

Still a further aspect of the present invention is the use of a delayed-release dosage form of a glucocorticoid for the manufacture of a medicament for the treatment of asthma in
(i) patients who have been pre-treated with an immediate release dosage form of a glucocorticoid,
(ii) patients who are refractory to treatment with an immediate release dosage form of a glucocorticoid, or
(iii) glucocorticoid naïve patients.

For example, the delayed-release form of a glucocorticoid may be administered to patients who have been previously treated with an oral immediate release dosage form of a glucocorticoid, e.g. a daily dose of 5-20 mg or 10-20 mg prednisone.

Still a further aspect of the present invention refers to the use of a delayed-release dosage form of a glucocorticoid for the manufacture of a medicament for the treatment of asthma in
(i) patients who have been pre-treated with other medicaments like inhaled glucocorticoids, β2-agonist, theophylline or a leukotriene antagonist or any combination thereof, or
(ii) patients who have not been pre-treated with any other medicaments like inhaled glucocorticoids, β2-agonist, theophylline or a leukotriene antagonist.

Still a further aspect of the present invention refers to the use of a delayed-release dosage form of a glucocorticoid for the manufacture of a medicament for the treatment of asthma in combination with at least one further medicament which is an inhaled glucocorticoids, β₂-agonist, theophylline or a leukotriene antagonist.

Still a further aspect of the present invention is the use of a delayed-release dosage form of a glucocorticoid for the manufacture of a medicament for the treatment of asthma without any further medicament.

Still a further aspect of the present invention is the use of a delayed-release dosage form of a glucocorticoid for the manufacture of a medicament for treatment of asthma in combination with reduced doses of at least one further medicament which is an inhaled glucocorticoids, β₂-agonist, theophylline or a leukotriene antagonist.

Still a further aspect of the present invention is the use of a delayed-release dosage form of a glucocorticoid for the manufacture of a medicament for the treatment of elevated inflammation parameters such as cytokines in asthma.

Still a further aspect of the present invention is a method for the treatment of a patient suffering from asthma, which comprises administering to said patient an effective amount of a glucocorticoid contained in a delayed-release dosage form, wherein said treatment is administered once daily for at least about two weeks.

Still a further aspect of the present invention is a method for the treatment of an asthma patient having daily fluctuations in cytokines due to underlying inflammation, which comprises administering to said patient an effective amount of a glucocorticoid contained in a delayed-release dosage form, wherein said treatment is administered once daily for at least about two weeks, and wherein said treatment is administered such that the glucocorticoid is released at or before the time when the patient's cytokine level is at a daily peak.

### DETAILED DESCRIPTION OF THE INVENTION

The present invention refers to the use of a delayed-release dosage form of a glucocorticoid. The release of the active ingredient is preferably delayed for a time period of 2-10 hours after intake, preferably 2-6, more preferably 3-5 hours after intake the active ingredient may be released in the upper sections of the intestine and/or in the lower sections of the intestine. More preferably, the active ingredient is released in the upper sections of the intestine within a period of 2-6 hours. The delayed-release dosage form is preferably administered to the patient at or before bedtime, more preferably in the evening, e.g. from about 9:00 pm to about 11:00 pm.

The delayed-release dosage form can be any kind of dosage form like a capsule or a tablet. It is preferably a tablet, e.g. as described in WO 2006/027266, which is herein incorporated by reference. The dosage form preferably comprises
(a) a core having at least one glucocorticoid-active ingredient and having at least one swellable adjuvant and/or a disintegrant such that the active ingredient is rapidly released from the dosage form when the core is contacted with gastrointestinal fluids, and
(b) an inert, e.g. a non-soluble and non-swellable coating pressed onto the core, said coating being capable of preventing substantial release of the active ingredient for a defined time period following ingestion of the dosage form.

The inert coating initially prevents release of the active ingredient or the active ingredient combination over an exactly defined period, so that no absorption can occur. The water present in the gastrointestinal tract penetrates slowly in through the coating and, after a time which is previously fixed by the pressure for compression, reaches the core. The coating ingredients show neither swelling nor diluting of parts of the coating. When the core is reached, the water penetrating in is very rapidly absorbed by the hydrophilic ingredients of the core, so that the volume of the core increases greatly or disintegrates and, as a consequence thereof, the coating completely bursts open, and the active ingredient and the active ingredient combination respectively is released very rapidly.

A particularly advantageous embodiment of this press-coated delayed-release tablet is achieved when a previously compressed core tablet is subsequently compressed with a multilayer tablet press to a press-coated tablet.

The tablet coating typically consists of the following materials in order to achieve a delayed release profile:
polymer or copolymer of acrylic acid, methacrylic acid etc. (e.g. Eudragits or Carbopol),
cellulose derivatives such as hydroxypropylmethylcellulose, hydroxypropylcellulose, carboxymethylcellulose, ethylcellulose, cellulose acetate,
polyvinyl alcohol,
polyethylene glycol,
salts of higher fatty acids, esters of monohydric or polyhydric alcohols with short-, medium- or long-chain, saturated or unsaturated fatty acids. Specifically, stearic acid triglycerides (e.g. Dynersan) or glycerol behenate (e.g. Compritol) are used.

In addition, further adjuvants should also be added to these materials so that the tablet coating can be compressed. Typically used here are fillers such as lactose, various starches, celluloses and calcium hydrogen phosphate or di-basic calcium phosphate. The glidant used is normally magnesium stearate, and in exceptional cases also talc and glycerol behenate. A plasticizer is often also added to the coating material, preferably from the group of polyethylene glycol, dibutyl phthalate, diethyl citrate or triacetin.

In order to achieve an optimal release profile, the tablet core must also fulfil certain tasks and exhibit certain properties. Thus, after the lag phase has elapsed, a rapid release profile is achieved if typical disintegrants are added to the inner core, which are derived for example from the group of the following substances: cellulose derivatives, starch derivatives, crosslinked polyvinylpyrrolidone. The use of a blowing agent, for example resulting from a combination of a weak acid and a carbonate or bicarbonate, may also promote rapid release. The tablet core typically consists additionally of matrix or filling ingredients (e.g. lactose, cellulose derivatives, calcium hydrogen phosphate or other substances known from the literature) and lubricant or glidant (usually magnesium stearate, in exceptional cases also talc and glycerol behenate).

The size of the core tablet preferably should not exceed 6 mm (preferably 5 mm) in diameter, because otherwise the press-coated tablet becomes too large for convenient ingestion. As a result thereof, the dosages of the active ingredients are in the range from 0.1 to 50 mg, very particularly between 1 and 20 mg.

The in vitro release profile of the dosage form according to the invention is preferably such that less than 5% of the active ingredient is released during the lag phase. After the release phase has started, preferably ≧80%, particularly preferably ≧90%, of the active ingredient is released within one hour. More preferably, the delayed-release dosage form has a dissolution time of equal to or less than about 2 hours after the lag time has been reached). The in vitro release is preferably determined using the USP paddle dissolution model in water.

The employed active ingredients are derived from the group of glucocorticoids and all show comparable physicochemical properties. Such include cortisone, hydrocortisone, prednisone, prednisolone, methylprednisolone, budesonide, dexamethasone, fludrocortisone, fluocortolone, cloprednole, deflazacort, triamcinolone, or the corresponding pharmaceutically acceptable salts and/or esters thereof. This applies in particular to prednisone, prednisolone, methylprednisolone, budesonide, dexamethasone, fluocortolone, cloprednole, and deflazacort or the corresponding pharmaceutically acceptable salts and/or esters thereof.

In the present case of the delayed-release tablet, the following combination of core materials and coating materials has proved to be particularly suitable for achieving a time- and site-controlled release with exclusion of pH and food influences:

The coating preferably comprises:
hydrophobic, waxy substances with an HLB value of less than about 5, preferably around 2. Carnauba wax, paraffins, cetyl ester waxes are preferably employed therefor. Glycerol behenate has proved to be particularly suitable. The use of about 20-60%, in particular about 30-50%, in the coating has proved to be very advantageous;
non-fatty, hydrophobic filling materials such as calcium phosphate salts, e.g. dibasic calcium phosphate. The use of about 25-75% of these filling materials, in particular of about 40-60%, in the coating has proved to be very advantageous here;
in addition, the tablet coating preferably also consists of binders, e.g. polyvinylpyrrolidone (PVP), typically in concentrations of about 4-12%, specifically about 7-10%, and glidants such as magnesium stearate, in concentrations of about 0.1-2%, in the specific case of about 0.5-1.5%. Colloidal silicon dioxide can for example be used as flow regulator, normally in concentrations of about 0.25-1%. In addition, to distinguish different dosages, a colorant can be added to the tablet coating, preferably an iron oxide pigment in concentrations of about 0.001-1%.

The core tablet preferably comprises:
an active ingredient or an active ingredient combination from the group of glucocorticoids, preferably prednisone, prednisolone, methylprednisolone, budesonide, dexamethasone, fiudrocortisone, fluocortolone, cloprednole, deflazacort, and triamcinolone, and the corresponding salts and esters thereof. The dosages of the active ingredients are in the region of about 0.1-50 mg, very especially between about 1 and 20 mg;
in addition, the core tablet preferably comprises a filler such as, for example, lactose, starch derivatives or cellulose derivatives. Lactose is preferably employed. The filler is typically present in concentrations of about 50-90%, specifically of about 60-80%. A disintegrant is additionally present and is typically crosslinked PVP or sodium carboxymethylcellulose, typically in concentrations of about 10-20%. It is additionally possible for a binder, e.g. PVP, to be present, typically in concentrations of about 2-10%, specifically of about 5.5-9%, and a lubricant such as magnesium stearate, in concentrations of about 0.1-2%, in the specific case of about 0.5-1.5%. Colloidal silicon dioxide is normally used as flow regulator, normally in concentrations of about 0.25-1%. It is additionally possible, for visually distinguishing the core from the coating, to add a colorant, preferably an iron oxide pigment in concentrations of about 0.01-1%.

In an especially preferred embodiment the delayed-release dosage form is Lodotra® comprising prednisone as an active ingredient.

Preferably, the delayed-release dosage form is administered as a long-term treatment to a subject in need thereof for a time sufficient to reduce and/or abolish the disease and/or disease symptoms. The long term treatment usually comprises daily administration of the medicament for an extended period of time, e.g. for at least two weeks, preferably for at least 4 weeks, more preferably for at least 8 weeks, even more preferably for at least 12 weeks, and most preferably for at least 6 months or at least 12 months.

According to the present invention refers to the novel treatment of groups of patients suffering from asthma.

These patient groups are selected from:
(i) patients with poor asthma related quality of life. i.e. medium to low AQLQ scores, e.g. 1-4
(ii) patients with moderate to poor asthma control, i.e. medium to high ACQ scores, e.g. 4-7 or 5-7
(iii) patients with medium to high number of nocturnal awakenings, e.g. ≧1 or ≧5 awakenings per week
(iv) patients with short disease duration of less than 2 years,
(v) patients with mid-term disease duration of 2-5 years, and
(vi) patients with long-lasting disease duration of more than 5 years.

For example, administration of delayed-release glucocorticoid dosage forms may lead to an increase of Asthma Quality of Life Questionnaire (AQLQ) scoring to at least 4, preferably at least 4.5 and most preferably at least 5.0 after 4 weeks administration of the delayed-release glucocorticoid dosage form.

For example, administration of the delayed-release glucocorticoid dosage form may lead to a decrease of Asthma Control Questionnaire (ACQ) scoring to 2.5 or less, preferably to 2.0 or less, more preferably to 1.5 or less and most preferably to 0.5 or less after 4 weeks administration of the delayed-release glucocorticoid dosage form.

Preferably, administration of the delayed-release glucocorticoid dosage form leads to a reduction of nocturnal awakenings to 2 or less, more preferably to 1 or less and most preferably to 0 awakenings per week after 4 weeks administration of the delayed-release glucocorticoid dosage form.

Further patient groups may be selected from:
(i) patients who have been pre-treated with an immediate release dosage form of a glucocorticoid;
(ii) patients who are refractory to treatment with an immediate-release dosage form of a glucocorticoid, and
(iii) glucocorticoid naive patients.

Further patient groups may be selected from:
(i) patients who have been pre-treated with other medicaments like an inhaled glucocorticoid, β2-agonist, theophylline or a leukotriene antagonist or any combination thereof, and
   patients who have not been pre-treated with any other medicaments like an inhaled glucocorticoids, β2-agonist, theophylline or a leukotriene antagonist.

By means of administering a delayed-release tablet, the daily dose of the glucocorticoid may be substantially reduced compared to an immediate-release tablet of the glucocorticoid. Thus, the disease-inhibiting effect may be obtained by a significantly lower dose of the active ingredient, whereby the occurrence and/or intensity of site effect is diminished. For example, the daily dose of the glucocorticoid can be reduced by at least 10%, more preferably by at least 20%, e.g. by 10-50% compared to an immediate-release tablet.

The treatment according to the present invention may comprise the treatment of asthma without any further medicament. On the other hand, the invention may comprise the treatment of asthma in combination with at least one further medicament which is preferably selected from the groups of inhaled glucocorticoids, short and long acting β₂ adrenergic receptor agonists, leukotriene antagonists, theophylline or combinations thereof.

The dose of the at least one further medicament may be substantially reduced e.g. by at least 10%, preferably by at least 20%, e.g. by 10-50%.

The present invention particularly refers to the treatment of asthma. Based on the results of the clinical trial described in the present application, it is evident that the delayed-release dosage form of a glucocorticoid, is of therapeutic benefit.

The dose of the glucocorticoid may vary during the course of treatment. For example, the patient may be administered a relatively high dose during the initiation of therapy (e.g., about 5-100 mg/day or higher of prednisone, or an equivalent amount of another glucocorticoid), which may be reduced downward over a period of time (e.g., over 3-4 weeks) according to the patient's response, to a maintenance therapy dose of about 1-50 mg/day or less, particularly 1-10 mg/day of prednisone, or an equivalent amount of another glucocorticoid. Alternatively, the patient may be started on a relatively low dose, which may be adjusted upward over a period of time (e.g., over 3-4 weeks) to a maintenance therapy dose of about 1-50 mg/day or less, particularly 1-10 mg/day of prednisone, or an equivalent amount of another glucocorticoid.

In a particular embodiment of the present invention the pharmacokinetic behaviour after administering the delayed-release dosage form is equivalent to the pharmacokinetcis after administering an immediate-release glucocorticoid dosage form, particularly a formulation of the same dosage of the same glucocorticoid. An equivalent pharmacokinetic behaviour may include an equivalent maximum plasma concentration (Cmax), i.e. a Cmax which is about 80 to about 125%, more particularly about 90 to about 110% of the Cmax of the corresponding immediate-release formulation. An equivalent pharmacokinetic behaviour may also include an equivalent AUC, which may be about 80 to about 125%, particularly about 90 to about 110% of the AUC of the corresponding immediate-release formulation. Further, the equivalent pharmacokinetics may include an equivalent tmax-tlag value for the delayed release and the immediate release formulation, particularly about 1 to 4 hours, more particularly about 2 to 3 hours, wherein tmax is the time after administration when Cmax is reached. Tlag corresponds to the in vivo lag-time for the release of the delayed-release dosage form. For an immediate-release dosage form, the value of tlag is about 0 h. The value tmax-tlag may be between about 2 and 3 hours. Further, the value tmax-tlag may be independent from the administered dosage of the glucocorticoid.

The delayed-release dosage form is advantageously administered together with or, e.g., not later than 3 h after a meal, e.g. during or upon 3 h after a meal.

Further, the present invention is described in more detail by the following figures and examples.

### FIGURE LEGENDS

FIG. 1 shows the pharmacokinetic profiles of Delayed-Release Prednisone and IR prednisone, Study NP01-013;

FIG. 2 shows pharmacokinetic profiles of Delayed-release Prednisone and IR prednisone, Study NP01-013, lag time corrected (tmax-tlag).

### EXAMPLES

Clinical studies. The clinical development program supporting the present application for the delayed-release prednisone tablet “Prednisone delayed-release” comprised 3 phase I studies and a pilot (phase IIa) study in the indication asthma:

### Phase I Studies:

EMR 62215-001 and EMR 62215-002 were conducted to investigate the bioavailability and pharmacokinetic characteristics of experimental Delayed-Release Prednisone formulations with the aim to select a Delayed-Release Prednisone tablet with appropriate pharmacokinetic profile for evening administration.
EMR 62215-005 was conducted to compare the bioavailability and pharmacokinetic characteristics of Delayed-Release Prednisone (5 mg, administered in the evening) with immediate-release prednisone (5 mg, administered at 2 am).
NP01-006 evaluated the food effect.
NP01-008 evaluated the dose proportionality of 1 mg, 2 mg and 5 mg tablets.
NP01-009 and NP01-010 evaluated the bioavailability of batches with different in vitro lag times, NP01-013 compared the bioavailability of Delayed-Release Prednisone (5 mg, administered at 10 pm after a light evening meal) and an IR prednisone formulation (5 mg, administered in the morning after breakfast).
   Phase IIa pilot study (asthma): in this open-label, explorative study the final prednisone modified-release (MR) tablet formulation was administered in the evening for 4 weeks. Efficacy and safety were compared to a preceeding 4 week period during which the same amount of reference IR product was given.

### Study Design and Methodology

### Pharmakokinetic Studies

NP01-006 (5 mg delayed-release prednisone; Food effect study): For prednisone no food effect has been reported in the literature. Prokein (1982) compared overnight fast vs. fed with 3 different diets and could not show any difference. He confirmed the findings from Tembo (1976) and Uribe (1976).

Surprisingly, in Study NP01-006 for Delayed-Release Prednisone a distinct effect of food on the oral bioavailability was shown.

In a study with 24 healthy subjects, oral absorption of prednisone from delayed-release prednisone was significantly affected by the intake of food. Under standard fasting conditions, both the maximum plasma concentration (Cmax) and the bioavailability of delayed-release prednisone were significantly lower than under fed conditions, shortly after intake of a high fat breakfast. The results are shown in Table 1. However, the amount of food and the timing of the meal relative to drug intake do not have an impact on the bioavailability of Delayed-Release Prednisone: both formulations where found to be bioequivalent when Delayed-Release Prednisone was taken 0.5 hour after a full meal or 2.5 hours after a light meal. Delayed-release prednisone thus should be taken not later than 3 h after a meal.

TABLE 1
Effect of Food on Delayed-Release Prednisone
Pharmacokinetics. Mean (SD)

| | | Delayed-Release | Delayed-Release |
|---|---|---|---|
| | | Prednisone | Prednisone |
| Prednisone | N | Fasted | Fed |
| Cₘₐₓ (ng/mL) | 24 | 6.6 (3.7) | 19.1 (3.2) |
| AUC₀₋ₗₐₛₜ (ng h/mL) | 24 | 34.2 (21.9) | 100.8 (18.7) |
| AUC_{0-∞} (ng h/mL) | 24 | 38.3 (21.8) | 103.0 (18.9) |
| t_{lag} (h) | 24 | 5.5 (3.5-7.5) | 4.5 (3.5-6.0) |
| tₘₐₓ (h) | 24 | 8.0 (6.0-18.0) | 6.5 (5.5-10.0) |
| t_{1/2} (h) | 24 | 2.6 (1.1) | 2.5 (0.5) |

tₘₐₓ and t_{lag} values are median (range)

NP01-013 compared the bioavailability of Delayed-Release Prednisone (5 mg, administered at 10 pm after a light evening meal) and Immediate Release prednisone (5 mg, administered in the morning after breakfast).

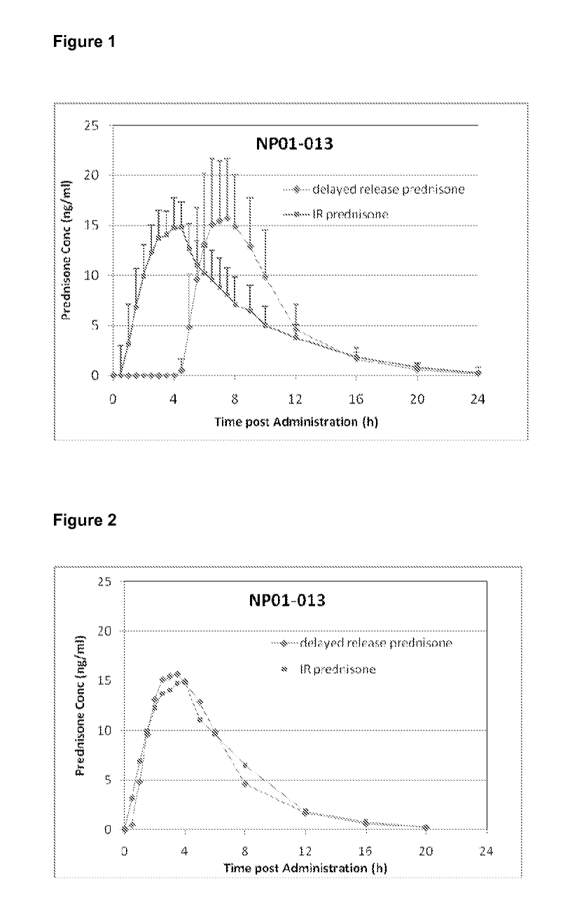

Surprisingly, the shape of the plasma-profile of both tablets, Delayed-Release Prednisone and Immediate release prednisone, were similar after the lag time has been achieved for the Delayed-Release Prednisone: Cmax, AUC and tmax-tlag were comparable. Tlag describes the lag time in vivo, Tmax describes the time until Cmax in reached. Surprisingly, tmax-tlag was for both Delayed-Release Prednisone and Immediate release prednisone about 2-4 hours. A further surprising finding was that the plasma profiles were identical under the concomitant administration of food. The results are shown in FIGS. 1 and 2.

### Phase IIa Study

### Methodology:

This trial is a single-centre, open label, phase IIa, single-treatment arm explorative study. After a 4 week run-in period, patients will be switched to an identical dose of modified-release prednisone (Lodotra®) and treated for another 4 weeks.

### Study Period:

Planned duration of the study (for each patient):10 weeks (including a 2-week screening period).

### Objectives:

Primary objective of the study: to evaluate, in subjects suffering from glucocorticoid-dependent persistent asthma, the efficacy and safety of Lodotra administered by oral route at 10 pm on the nocturnal symptoms of asthma and on respiratory function, compared to the usual ad-ministration of the equivalent dose of immediate-release prednisone (ad-ministered at 8 am).
Secondary objective of the study: to investigate the safety and tolerability of the modified-release tablet formulation of prednisone
   Number of patients:

The objective of this exploratory, proof-of-concept study is to collect safety and efficacy data in order to carry out a controlled study at a later date. The formal calculation of the number of patients necessary for the study was therefore not performed. A minimum of ten and a maximum of twenty evaluable patients completing both study periods will be included.

### Diagnosis and Criteria for Inclusion:

The study population will be made up of asthmatic patients aged at least 18 years, suffering from severe persistent asthma, having nocturnal symptoms (at least 3 nocturnal awakenings due to asthma during the last screening week) and a treatment by oral glucocorticoids.

To be eligible for the study, patients must meet all the following criteria:
The subject must be able to understand the terms of the written in-formed consent form, and must provide a dated and signed form before the start of any study procedure
Aged at least 18 years
Patient having a diagnosis of asthma dating back more than 6 months at the time of inclusion
Asthma necessitating a continuous treatment by oral corticoids
A minimum of 3 nocturnal awakenings due to asthma during the last screening week
Stable dose of oral glucocorticoids for at least 4 weeks prior to inclusion into the study
No change in asthma medication during the last 4 weeks prior to V0
Non-smoker or ex-smoker (having stopped smoking more than one year previously and with a smoking history of less than 10 pack years)
Female patients of childbearing potential must be using a medically accepted contraceptive regimen
Able to perform the required study procedures including handling of medication containers and diaries.

### Exclusion Criteria:

The presence of any of the following will exclude a patient from enrolment in the study:
Poorly controlled asthma, defined as meeting at least one of the following within the 4 weeks prior to Visit V0:
   hospital admission for asthma (including treatment in an emergency room),
   a lower airway infection,
Diagnosis of chronic obstructive pulmonary disease or other relevant lung diseases (e.g. history of bronchiectasis, cystic fibrosis, bronchiolitis, lung resection, lung cancer, active tuberculosis, interstitial lung disease)
Clinically significant abnormalities of the hematological or bio-chemical constants
Pregnancy or breastfeeding
Participation in another clinical study within 30 days preceding Visit V0,
Re-entry of patients previously enrolled in this trial,
Suspected inability or unwillingness to comply with the study procedures
Alcohol or drug abuse
Need to take a non-authorised concomitant treatment (cf. list of medicaments not authorised during the study) in the course of the study
Other disease requiring treatment with corticosteroids
Subject is the investigator or any subinvestigator, research assistant, pharmacist, study coordinator, other staff or relative thereof directly involved in the conduct of the protocol
Patient with a hospitalisation scheduled during the study period
Any uncontrolled concomitant disease requiring further clinical evaluation (e.g. uncontrolled diabetes, uncontrolled hypertension, etc.).

### Criteria for Entering the Treatment Period:

Patients must meet all of the following criteria to be eligible for entering the treatment period with modified-release prednisone at visit V2:
Compliance in filling in the patient diary. Patient compliance must be checked on the dates of visits and will be considered sufficient if the deviation does not exceed 3 days and if compliance with the treatment is maintained. Wider deviations must be corrected at the sequential visits in order to adhere to the total duration of the study
Absence of asthma exacerbation during the run-in period (an exacerbation is defined by an increase in the corticoid dose, an emergency consultation or a hospitalisation for asthma).

### Duration of Treatment:

Run-in period: 4 weeks
Treatment period: 4 weeks

### Test Product, Dose and Mode of Administration:

5 mg modified-release tablet formulation of prednisone (Lodotra®) and/or
1 mg modified-release tablet formulation of prednisone (Lodotra®)

### Reference Product:

immediate release prednisone

### Dosing:

During run-in: at 8 am: immediate release prednisone tablets

During treatment: at 10 pm: modified-release prednisone tablets

### Concomitant Medication:

Not allowed:
oral or parenteral glucocorticoids other than the study medication

### Allowed:

Asthma medication according to patients' individual needs on a stable dose.
Other drugs for the treatment of concomitant diseases are authorised. However, their dosage should be kept constant throughout the study.

### Criteria for Evaluation:

### Efficacy:

### Primary Endpoint

Variation in the total number of nocturnal awakenings between the last 2 weeks of run-in and the last 2 weeks of treatment by Lodotra.

### Secondary Endpoint

Variation in the total number of nocturnal awakenings between:
the last week of run in and the last week of treatment by Lodotra
the all run in period and the all treatment by Lodotra period.
Variation of PEF (l/min) in the morning between the last 2 weeks of run-in and the last 2 weeks of treatment by Lodotra.
Variation of PEF (l/min) in the evening between the last 2 weeks of run-in and the last 2 weeks of treatment by Lodotra
Variation between visit 2 and visit 4 of the spirometric variable FEV1, FVC, PEF, and exhaled NO.
Variation between the last 2 weeks of run-in and the last 2 weeks of treatment by Lodotra in the score of the asthma symptoms and the use of rescue medication.
Variation between the visit V2 and the visit V4 in ACQ/AQLQ questionaires
Description of the use of inhaled steroids and treatment for severe asthma exacerbations

### Safety:

### Safety is Evaluated by:

Adverse events (collected at each visit)
New clinical signs at the physical examination (collected at each visit)
Changes in vital signs or biological data (collected during the administration of the study product).

### Study Results

### Baseline Characteristics

### Efficacy Results

The primary efficacy endpoint of the study was the number of nocturnal awakenings due to asthma symptoms. The first 5 patients that completed the study, showed a clinically relevant reduction of the number of nocturnal awakenings due to asthma, after being switched from standard immediate release prednisone to an identical dose of Lodotra. The results are shown in Table 2.

TABLE 2
Number of nocturnal awakenings due to asthma under
standard immediate release prednisone and Lodotra

| | |
|---|---|
| Standard IR | Lodotra |

| 2 week period | 1 to 2 | 3 to 4 | 5 to 6 | 7 to 8 |
|---|---|---|---|---|
| Patient 01 (20 mg) | 28 | 15 | 9 | 1 |
| Patient 02 (45 mg) | 23 | 14 | 7 | 0 |
| Patient 03 (5 mg) | 17 | 12 | 7 | 12 |
| Patient 04 (10 mg) | 9 | 4 | 0 | 0 |
| Patient 05 (10 mg) | 6 | 1 | 4 | 0 |

In addition, after a switch to treatment with Lodotra, all 5 patients achieved a clinically meaningful better control of their asthma, measured by the Asthma Control Questionnaire (ACQ) (Juniper 1999). A change in score of 0.5 is the smallest change that can be considered clinically important and would justify a change in patients' treatment. Patients with a score below 0.5 are considered controlled. The results are shown in Table 3.

TABLE 3
Asthma control scores (ACQ) under standard
immediate release prednisone and Lodotra

| | |
|---|---|
| Standard IR | Lodotra |

| 2 week period | 1 to 2 | 3 to 4 | 5 to 6 | 7 to 8 |
|---|---|---|---|---|
| Patient 01 (20 mg) | 4.5 | 3.0 | 2.8 | 1.7 |
| Patient 02 (45 mg) | 3.2 | 3.3 | 2.7 | 1.3 |
| Patient 03 (5 mg) | 3.5 | 3.2 | 1.5 | 2.2 |
| Patient 04 (10 mg) | 3.2 | 3.2 | 2.3 | 2.3 |
| Patient 05 (10 mg) | 4.2 | 3.2 | 1.5 | 2.5 |

The observed improvements in clinical endpoints clearly resulted in a clinically relevant improvement in asthma related quality of life measured by the AQLQ (Juniper.1992). The results are shown in Table 4.

TABLE 4
Asthma related quality of life (AQLQ) under
standard immediate release prednisone and Lodotra

| | |
|---|---|
| Standard IR | Lodotra |

| 2 week period | 1 to 2 | 3 to 4 | 5 to 6 | 7 to 8 |
|---|---|---|---|---|
| Patient 01 (20 mg) | 2.6 | 3.2 | 3.6 | 4.9 |
| Patient 02 (45 mg) | 3.1 | 3.5 | 4.3 | 5.0 |
| Patient 03 (5 mg) | 4.6 | 4.5 | 6.2 | 6.0 |
| Patient 04 (10 mg) | 4.3 | 4.4 | 5.1 | 4.6 |
| Patient 05 (10 mg) | 2.9 | 3.9 | 4.8 | 4.1 |

The minimally clinically important difference is 0.5 on the 7-point scale, where higher scores means a better quality of life. All 5 patients achieved a clinically relevant improvement after being switched to an identical dose of Lodotra.

### Benefits and Risks Conclusions

Prednisone delayed-release is a novel, delayed-release tablet that has been developed to optimize the efficacy of orally administered prednisone in the treatment of chronic inflammatory diseases such as RA and asthma. Prednisone delayed-release has shown clinically relevant improved efficacy compared to standard prednisone in patients with asthma without increasing their prednisone dose. This improvement has been solely obtained as a result of Prednisone delayed-release's unique release characteristics. The safety profiles of Prednisone delayed-release and standard prednisone were comparable and the patients were thus not exposed to an increased risk.

The benefits and main features of Prednisone delayed-release can be summarized as follows:

A significant decrease in the numbers of nocturnal awakenings due to asthma along with clinically relevant improvements in asthma symptom control and asthma related quality of life was obtained in patients with long-standing asthma who were pretreated with oral prednisone and with inhaled glucocorticoids and short and long acting β₂ adrenergic receptor agonists and other asthma medications.

Maximum plasma levels of prednisone in the early morning hours are obtained by administration of Prednisone delayed-release at about 22:00 which is an acceptable time for the patient.

Prednisone delayed-release tablets can be used in patients with severe or moderate disease.

Prednisone delayed-release tablets can be used in patients with short, mid-term or long-lasting disease duration.

Prednisone delayed-release tablets can be used in patients pre-treated with corticosteroids, in those who are refractory to treatment or in corticoid naïve patients.

Prednisone delayed-release tablets can be used as monotherapy or more likely in combination with inhaled glucocorticoids, short and long acting β₂ adrenergic receptor agonists, leukotriene antagonists or theophylline.

Prednisone delayed-release tablets can be used for short, mid or long-term treatment.

### LITERATURE REFERENCES

Beam et al.: Timing of prednisone and alterations in airways inflammation in nocturnal asthma (1992) Am Rev Respir Dis:146; 1524-30
Dethlefsen U, Repgas R: Ein neues Therapieprinzip bei nächtlichem asthma. Klein Med (1985): 80; 44-47
Global Strategy for Asthma Management and Prevention, Global Initiative for Asthma (GINA) 2008. Available from: http://www.ginasthma.org.
Holgate S, Polosa R: The mechanisms, diagnosis, and management of severe asthma in adults: Lancet (2006): 368; 780-793
Juniper E F et al.: Evaluation of impairment of health-related quality of life in asthma: development of a questionnaire for use in clinical trials. Thorax (1992) 47: 76-83.
Juniper E F et al.: Development and validation of a questionnaire to measure asthma control. Eur Respir J (1999) 14: 902-7
Niphadkar, Clinical Therapeutics (2005): 27; 11; 1752-1763
Sutherland R E Nocturnal asthma: J Allergy Clin Immunol (2005): 116; 1179-1186
Turner-Warwick M: Epidemiology of nocturnal asthma: Am J Med (1988) 85; 6-7
Zetterström, Respiratory Medicine (2008), 102, 1406-1411

1. A method for the treatment of a patient suffering from asthma, which comprises administering to said patient an effective amount of a glucocorticoid contained in a delayed-release dosage form, wherein said treatment is administered at least for 2 weeks.

2. The method of claim 1, wherein the asthma is reversible or non reversible upon brochodilatator administration.

3. The method of claim 1, wherein the asthma is uncontrolled.

4. The method of claim 1, wherein the asthma is severe nocturnal asthma.

5. The method of claim 1, wherein said patients are suffering from medium to frequent nocturnal awakenings.

6. A method for the treatment of a patient suffering from uncontrolled asthma, particularly from uncontrolled asthma with nocturnal awakenings, which comprises administering to said patient an effective amount of glucocorticoid contained in a delayed-release dosage form, wherein said treatment is administered at least for 2 weeks.

7. A method for the treatment of an asthma patient having circadian fluctuations in Interleukin 6 levels due to underlying inflammation, which comprises administering to said patient an effective amount of a glucocorticoid contained in a delayed-release dosage form, wherein said treatment is administered once daily, and wherein said treatment is administered such that the glucocorticoid is released at or before the time when the patient's Interleukin 6 level is at a daily peak.

8. The method of claim 7, wherein said peak Interleukin-6 level occurs during the night.

9. The method of claim 1, wherein the treatment comprises administration of the glucocorticoid for at least about two weeks, preferably for at least about four weeks, more preferably for at least about 3 months and most preferably for at least about 12 months.

10. The method of claim 1 wherein said patients are suffering from medium to low scoring of the Asthma Quality of Life Questionnaire (AQLQ).

11. The method of claim 1 wherein said patients are suffering from high to medium scoring of the Asthma Control Questionnaire (ACQ).

12. The method of claim 1, wherein said patients are suffering from frequent usage of emergency medication.

13. The method of claim 1, wherein the glucocorticoid dose is equal or less than about 20 mg/day of prednisone or an equivalent amount of another glucocorticoid for the initiation and maintenance of the therapy.

14. The method of claim 13, wherein the optimal glucocorticoid dose can be chosen by the combination of delayed release dosage forms of different strengths of said glucocorticoid.

15. The method of claim 14, wherein the different strengths of the delayed release dosage forms are 1 mg, 2 mg, 2.5 mg, 5 mg, 7.5 mg and/or 10 mg of prednisone or an equivalent amount of another glucocorticoid.

16. The method of claim 1, 6 or 7, wherein said patient has not previously been treated with an oral immediate release glucocorticoid, inhaled glucocorticoids, short and long acting β₂ adrenergic receptor agonists, leukotriene antagonists, theophylline or combinations thereof.

17. The method of claim 1, wherein said patient has previously undergone treatment with an inhaled glucocorticoids, short and long acting β₂ adrenergic receptor agonists, leukotriene antagonists, theophylline, or combinations thereof.

18. The method of claim 1, which further comprises administering to said patient an effective amount of inhaled glucocorticoids, short and long acting β₂ adrenergic receptor agonists, leukotriene antagonists, theophylline, or combinations thereof.

19. The method of claim 1, wherein said patient has previously undergone treatment with an oral immediate release dosage form of a glucocorticoid.

20. The method of claim 19, wherein said patient is refractory to said treatment with an oral immediate release dosage form of a glucocorticoid.

21. The method of claim 19, wherein the immediate release dosage form of a glucocorticoid is replaced by the delayed release dosage form.

22. The method of claim 1, wherein said delayed-release dosage form is more effective at the same dose of glucocorticoid compared to the administration of said glucocorticoid contained in an immediate release dosage form.

23. The method of claim 1, wherein the dosage of the glucocorticoid can be reduced by administering said delayed-release dosage form compared to the administration of a glucocorticoid contained in an immediate release dosage form by at least 20%.

24. The method of claim 1, wherein said treatment consists essentially of administering to said patient an effective amount of a glucocorticoid contained in a delayed-release dosage form, wherein said treatment is administered once daily.

25. The method of claim 1, wherein the delayed release glucocorticoid form is administered in the evening.

26. The method of claim 24, wherein the delayed release glucocorticoid form is administered between about 9:00 pm and about 11:00 pm.

27. The method of claim 1, wherein the delayed-release dosage form has an in vivo lag time (tlag) of from about 2 hours to about 6 hours after administration.

28. The method of claim 27, wherein the delayed-release dosage form more specifically has an in vivo lag time (tlag) of from about 3 hours to about 5 hours after administration.

29. The method of claim 1, wherein the delayed-release dosage form is administered with food.

30. The method of claim 1, wherein the delayed release dosage form is a tablet or a capsule.

31. The method of claim 30, wherein the delayed-release dosage form does not have an enteric coating and has a drug release behaviour which is independent of pH.

32. The method of claim 30, wherein the delayed-release dosage form comprises a non-soluble/non-swellable coating and a core comprising the active agent and a disintegrant and/or a swelling agent.

33. The method of claim 1, 6 or 7, wherein the glucocorticoid is cortisone, hydrocortisone, prednisone, prednisolone, methylprednisolone, budesonide, dexamethasone, fludrocortisone, fluocortolone, cloprednole, deflazacort, triamcinolone, or the corresponding pharmaceutically acceptable salts and/or esters thereof.

34. The method of claim 33, wherein the glucocorticoid is prednisone, prednisolone, methylprednisolone, dexamethasone, fluocortolone, cloprednole, and deflazacort or the corresponding pharmaceutically acceptable salts and/or esters thereof.

35. A method for the treatment of a patient suffering from severe uncontrolled asthma which comprises administering to said patient an effective amount of a glucocorticoid contained in a delayed-release dosage form, wherein the pharmacokinetics after administering of said dosage form are equivalent to the pharmacokinetics after administering an immediate release dosage form, wherein the pharmacokinetics include an equivalent Cmax, an equivalent AUC and/or an equivalent tmax-tlag.

36. The method of claim 35, wherein the dosage of the glucocorticoid is identical between the delayed release dosage form and the immediate release dosage form.

37. The method of claim 35, wherein tmax-tlag is between 1 and 4 hours.

38. The method of claim 35, wherein tmax-tlag is independent from the administered dosage.

39. The method of claim 35, wherein Cmax and AUC are linear dependent from the administered dosage between 0.1 to 10 mg prednisone or the equivalent amount of another glucocorticoid.

40. The method of claim 35, wherein said treatment consists essentially of administering to said patient an effective amount of a glucocorticoid contained in a delayed-release dosage form, wherein said treatment is administered once daily.

41. The method of claim 35, wherein the delayed release glucocorticoid form is administered in the evening.

42. The method of claim 35, wherein the delayed release glucocorticoid form is administered between about 9:00 pm and about 11:00 pm.

43. The method of claim 35, wherein the delayed-release dosage form has an in vivo lag time (tlag) of from about 2 hours to about 6 hours after administration.

44. The method of claim 35, wherein the delayed-release dosage form more specifically has an in vivo lag time (tlag) of from about 3 hours to about 5 hours after administration.

45. The method of claim 35, wherein the delayed-release dosage form is administered with food.

46. The method of claim 35, wherein the delayed release dosage form does a tablet or a capsule.

47. The method of claim 35, wherein the delayed-release dosage form does not have an enteric coating and has a drug release behaviour which is independent of pH.

48. The method of claim 35, wherein the glucocorticoid is cortisone, hydrocortisone, prednisone, prednisolone, methylprednisolone, budesonide, dexamethasone, fludrocortisone, fluocortolone, cloprednole, deflazacort, triamcinolone, or the corresponding pharmaceutically acceptable salts and/or esters thereof.

49. The method of claim 35, wherein the glucocorticoid is prednisone, prednisolone, methylprednisolone, dexamethasone, fluocortolone, cloprednole, and deflazacort or the corresponding pharmaceutically acceptable salts and/or esters thereof.
